(19) United States
(12) Patent Application Publication
(10) Pub. No.: US 2002/0198551 A1
(43) Pub. Date: Dec. 26, 2002

(54) Endoscopic tissue separator surgical device

(76) Inventors: Kevin Lee Grant, Litchfield, NH (US); Charles M. Grinnell, Groton, MA (US); Brian Daniel Tracey, Litchfield, NH (US)

Correspondence Address: BROMBERG & SUNSTEIN LLP, 125 SUMMER STREET, BOSTON, MA, 02110-1618, US

(21) Appl. No.: 10/161,094

(22) Filed: Jun. 3, 2002

Related U.S. Application Data
(63) Continuation-in-part of application No. 09/703,532, filed on Nov. 1, 2000.
(60) Provisional application No. 60/165,707, filed on Nov. 16, 1999.

(51) Int. Cl. A61D 1/02; A61B 17/22

(52) U.S. Cl. 606/159

(57) ABSTRACT

An endoscopic tissue separator surgical device and method. The device has a multi-lumen shaft having proximal and distal ends, a central lumen for accepting an endoscope, and at least two fluid lumens, with a head coupled to the distal end of the shaft and a handle coupled to the proximal end. The head has an endoscope port and at least two fluid ports whose centers are all disposed along an arcuate line of curvature, while the handle has at least two fluid supply ports. Gas and fluid may be conveyed through the shaft from the handle to the head in the at least two fluid lumens separate from the lumen for accepting an endoscope. At least one lumen of the multi-lumen shaft may house a stainless steel tube with an inside diameter of sufficient size to accept an endoscope.

### CROSS REFERENCE TO RELATED APPLICATIONS

This application is a continuation-in-part of U.S. application Ser. No. 09/703,532, filed Nov. 1, 2000, which in turn claims priority from U.S. Provisional Application, Serial No. 60/165,707 filed Nov. 16, 1999, both of which are incorporated herein by reference.

### TECHNICAL FIELD AND BACKGROUND ART

The present invention relates to surgical devices and procedures, and more particularly to the separation and/or extraction of material during surgical procedures.

For example, excessive plaque build-up within arteries decreases the blood flow capacity of the arteries and of the living tissue supplied by the arteries. Normal blood flow may be restored by either removing the plaque build-up or by bypassing the blocked section of the artery. Both procedures require incisions along the blockage and become extremely invasive for extensive blockages that may run the length from the groin to the knee, for example.

A less invasive procedure for removing plaque build-ups utilizes a catheter which is configured to either grab or loosen the plaque build-up along the blockage as the catheter is passed through the blockage. This procedure requires two incision sites—one above the blockage and one below. Although the catheter will remove sufficient blockage to restore normal flow, the procedure may not remove all the build-up, and there exists a risk that the catheter head may penetrate through the plaque build-up layer and damage the adventitia layer of the artery. In addition, the procedure may worsen the blockage of the side branches by a “snowplow” effect as the catheter is pushed through the artery.

Instead of pushing a catheter through the blood flow channel of the artery, gas endarterectomy uses a gas to separate the media layer surrounding the blockage from the adventitia layer of the artery. Although the gas endarterectomy procedure also requires two incisions, the procedure does not suffer from the “snowplow” effect of the catheter procedure and may also remove the side branch plugs along with the main blockage. In addition, all plaque is removed in a gas endarterectomy procedure because the underlying intima and media layers containing the plaque are removed.

In U.S. Pat. No. 5,954,713 ('713) issued on Sep. 21, 1999 to Newman, et al., a gas endarterectomy procedure is described wherein only one incision point is required. As described, however, the procedure described in the '713 patent requires two instruments: a spatula instrument and a cutting instrument. Furthermore, both instruments must be worked through the blockage. This increases the duration of the requisite operation and the risk of damage to the adventitia layer of the artery. It is thus preferable to have a single instrument that both separates the media layer from the adventitia layer and then removes the blockage.

### SUMMARY OF THE INVENTION

An improved endoscopic tissue separator surgical instrument is provided, that may advantageously cause less potential damage to the underlying tissue than provided by earlier technologies. In accordance with embodiments of the invention, a design for the spatula head, shaft, and switch is described, resulting in an endoscopic tissue separator instrument that, for example, can readily slide between the media and intima layer and the adventitia layer of a blocked artery, without excessive stretching of the adventitia layer.

The endoscopic tissue separator surgical instrument provided in accordance with preferred embodiments of the present invention comprises a multi-lumen shaft, a head having an endoscope port and at least two fluid ports, a handle that includes a gas supply port and a fluid supply port in fluid communication with the at least two fluid ports of the head. In another embodiment in accordance with the present invention, the handle may further comprise a flow valve for metering flow of gas between the gas supply port and the at least two fluid ports on the head. In the endoscopic tissue separator according to embodiments of the present invention, the centers of the endoscope port and fluid ports are disposed along an arcuate line of curvature. The shaft has a proximal end and a distal end wherein the handle is coupled to the proximal end. In one embodiment, an endoscope may be connected to the endoscope port. In such an embodiment, the endoscope provides optical coupling between the distal and proximal ends of the shaft.

In accordance with alternate embodiments of the invention, the endoscopic tissue separator may also have a fluid inlet coupled to the handle for coupling a fluid supply line and thereby flow of saline solution or other fluid to a first fluid port on the head, and a gas inlet coupled to the handle for coupling a gas supply line and thereby flow of gas to a second fluid port on the head. Fluid connection of the handle to the head of the shaft may be provided through a first and second lumen, while an endoscope may provide optical coupling through a third lumen between the distal and proximal ends of the shaft. Furthermore, the centers of the endoscope port and the fluid ports, and the centers of the endoscopic lumen and fluid lumens, are disposed along an arcuate line of curvature.

The instrument may also have a grasping device, such that the tissue separator can thus also be used as a tissue extractor. The grasping device has a retracted configuration and a deployed configuration wherein the grasping device may be a barb or a hook and extends away from the head in the deployed configuration. Furthermore, there is a deployment control disposed on the handle of the instrument that is in mechanical communication with the grasping device and which operates as a slide substantially collinear with the shaft. The mechanical communication between the deployment control and grasping device includes a control wire connected to both the grasping device and the deployment control wherein the wire between the two is straight and is further connected to the deployment control by means of a mechanical capture.

In accordance with a further embodiment of the invention, there is provided a tissue separator and extractor surgical instrument that has a multi-lumen shaft with proximal and distal ends and a head coupled to the distal end of the shaft. The head has an endoscope port and at least two fluid ports such that the centers of the endoscopic port and fluid ports are disposed along an arcuate line of curvature. At least one lumen of the multi-lumen shaft houses a stainless steel tube with an inside diameter of sufficient size to accept the endoscope. A handle, coupled to the proximal end of the shaft, has at least two fluid supply ports in fluid communication with the at least two fluid ports on the head.

### BRIEF DESCRIPTION OF THE DRAWINGS

The foregoing features of the invention will be more readily understood by reference to the following detailed description, taken with reference to the accompanying drawings, in which:

FIG. 1 show a perspective view of an embodiment of the present invention.

FIG. 2 shows an improved head design of one embodiment of the present invention inserted into a blood vessel in an end-on cross sectional view;

FIG. 2b shows an existing head design of another embodiment of the present invention;

FIG. 3 shows a top view cross section of the improved head embodiment shown in FIG l;

FIG. 4 shows a cross-sectional view of an improved multi-lumen shaft design of one embodiment of the present invention;

FIG. 5 shows an isometric view of the device of one embodiment of the present invention;

FIG. 6 shows a cross-sectional view of an improved extractor switch of one embodiment of the present invention;

FIG. 7 shows a longitudinal cross-sectional view of the device of one embodiment of the present invention.

### DETAILED DESCRIPTION OF SPECIFIC EMBODIMENTS

Definitions. As used in this description and the accompanying claims, the following terms shall have the meanings indicated, unless the context otherwise requires:

Arc means a continuous curve having no inflections, including, for example, curves such as portions of a French curve, a hyperbola, and those with a circular angle of curvature.

Arcuate line of curvature means a line of curvature in the form of an arc, as defined above.

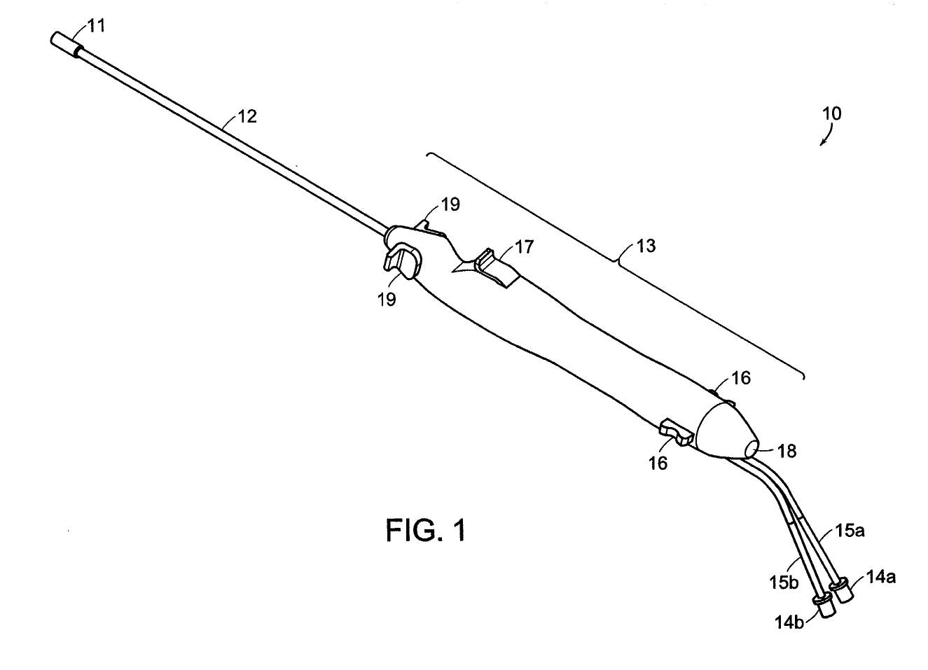

FIG. 1 shows a perspective view of an embodiment of the present invention. The tissue separator surgical instrument, generally designated by numeral 10 comprises a head 11, connected to a shaft 12, which in turn is connected to a handle 13. Handle 13 houses a first fluid port (not shown) that connects to a first fluid supply 15a with a female Leur connector 14a, and a second fluid port (not shown) connected to a second fluid supply line 15b with a female Leur connector 14b. A gas may be delivered to head 11 through supply line 15a or 15b, and delivery is controlled by a linear flow valve 17. The handle also houses an endoscope lock 16 to secure an endoscope to device 10, inserted into handle 13 via endoscope port 18. In addition, there is provided a deployment control 19 within handle 13 that controls deployment or retraction of a grasping device located in head 11, and which can be actuated from either side of handle 13. Handle 13 is connected to shaft 12 which is a relatively stiff, yet flexible tube providing multiple lumens as now described.

Shaft 12 is sized to provide flow paths both for the gas from the gas supply and for a saline solution to head 11 while also accommodating the endoscope and a control wire, not shown. The control wire is attached to a deployment control in handle 13, a slide in one particular embodiment, at one end and is attached to the grasping device located in head 11 at the other end. Head 11 is attached to the end of shaft 12 opposite the end attached to handle 13. Head 11 is provided with an opening that holds the end of the endoscope and also provides exit orifices for the gas and saline solution. Head 11 also contains a grasping device that may be deployed by the operation of deployment control 19.

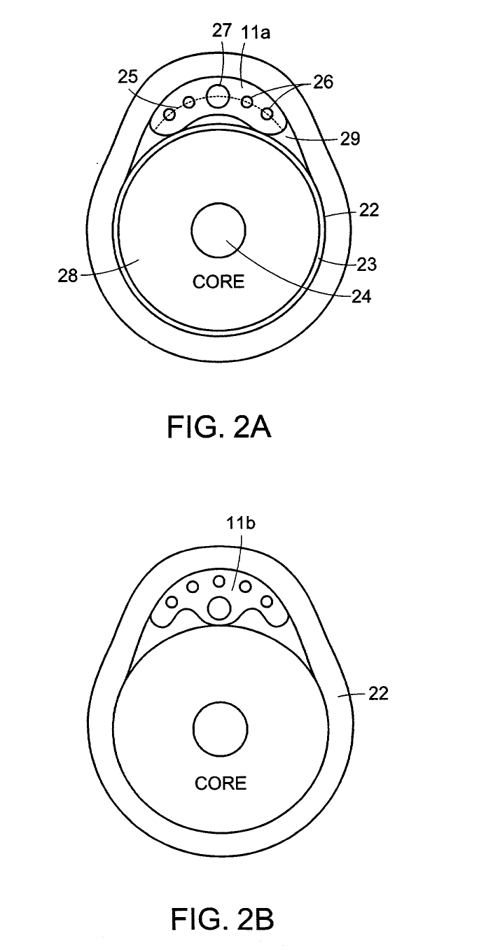

In accordance with one embodiment of the present invention, FIG. 2a shows a front view of an improved head design 11a inserted into an artery 20 shown in crosssection. Head 11 is pushed between the adventitia layer 22 of the artery and the media layer 23 of the artery. The media layer encircles the intima later, not shown, on which plaque forms a build-up layer 28 that obstructs the blood flow channel 24. New head design 11a comprises a central endoscope port 27 and at least two fluid ports 26 for fluid delivery of a gas and/or saline solution. Fluid ports 26 are in fluid communication with the gas flow path of shaft 12 (shown in FIG. 1.) through internal channels (not shown) within head 11a. Gas flowing through the fluid ports 26 separate the adventitia layer 22 from the media layer 23, creating an interstitial chamber 29 through which head 11a can travel along the blockage caused by the plaque build-up layer 28. Endoscope port 27 and the at least two fluid ports 26 are disposed along an arcuate line of curvature 25, thereby minimizing the overall thickness of head 11a. As shown in FIG. 2a, upon insertion into the artery, head design 11a requires less overall stretching of the adventitia layer 22 than does head design 11b, shown in FIG. 2b, which requires 13% more stretch of the adventitia layer 22.

It has been observed that the media and intima layer is fairly weak at the transition between the plaque build-up and no-plaque build-up regions. Therefore, if using an embodiment of the present invention that contains a grasping device, by grabbing the blockage and pulling, the blockage will tend to separate from the healthy media and intima layers at the transition without the use of a cutting tool. By eliminating the cutting tool, risk to the patient may be advantageously reduced; additionally, only the diseased portion of the media and intima layers need be removed. Furthermore, as described in U.S. application Ser. No. 09/703,532, filed Nov. 2, 2000, the break occurs at the point where the healthy media and intima layers are separated, thereby producing a smoother transition region.

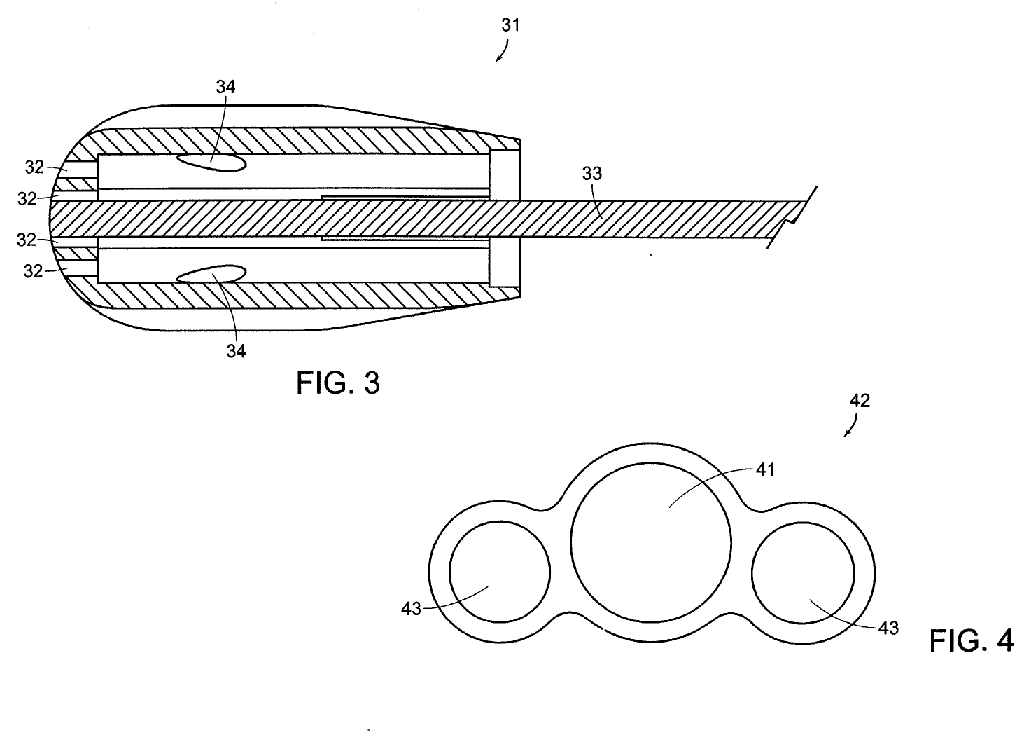

In accordance with some embodiments of the invention, spatula head 31 has multiple ports 32 and 34 as described with reference to FIG. 3. Ports 32 and 34 serve for delivery of gas and/or fluids, such as saline, into the interstitial space of the artery during tissue separation. An endoscope 33 runs through the center of head 31, and angled ports 34 may also be provided for extractor wires, not shown, to allow removal or plaque buildup within the artery.

FIG. 4 shows an expanded cross-sectional view of a multi-lumen shaft 12 in accordance with one embodiment of the present invention. A central lumen 41 for accommodating a tube of sufficient diameter to accept an endoscope is flanked by at least two additional lumens for the delivery of gas (typically carbon dioxide) and/or fluids to ports in the spatula head, for example ports 32 and 34 in spatula head 31 of FIG. 3. Lumen 41 accepts an endoscope and may be lined, for example, with a stainless steel tube or plug (not shown), which may advantageously provide stiffness, whether or not an endoscope is used.

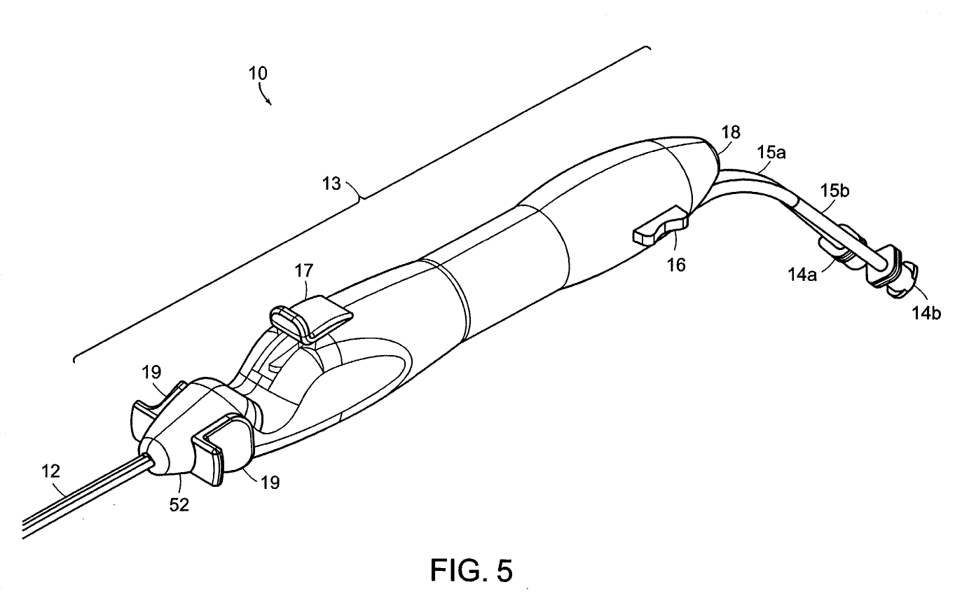

FIG. 5 shows an isometric view of an embodiment of the present invention, designated generally as 10. In this particular embodiment, shaft 12 is connected to handle 13 via and deployment controls 19 that may be actuated from both a left and a right side of handle 13. Gas and/or fluid is delivered to the head of shaft 12 through first and second fluid supply lines 15a and 15b which typically have Leur connectors of distinguishing polarity for connection to gas and fluid supplies. Handle 13 has an endoscope lock 16 to secure an endoscope to device 10. Delivery of gas through one of fluid lines 15a or 15b is controlled by linear flow valve 17.

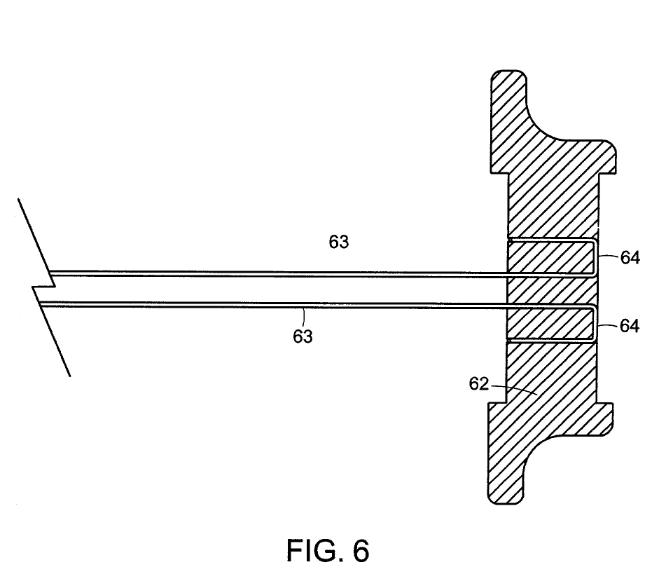

FIG. 6 shows a cross-sectional view of extractor switch 62. Control wires 63 run through shaft 12 to head 11, not shown, and are connected to extractor switch 62 by a mechanical capture in the form of U-shaped ends 64, in lieu of, or in addition to, standard adhesive attachment.

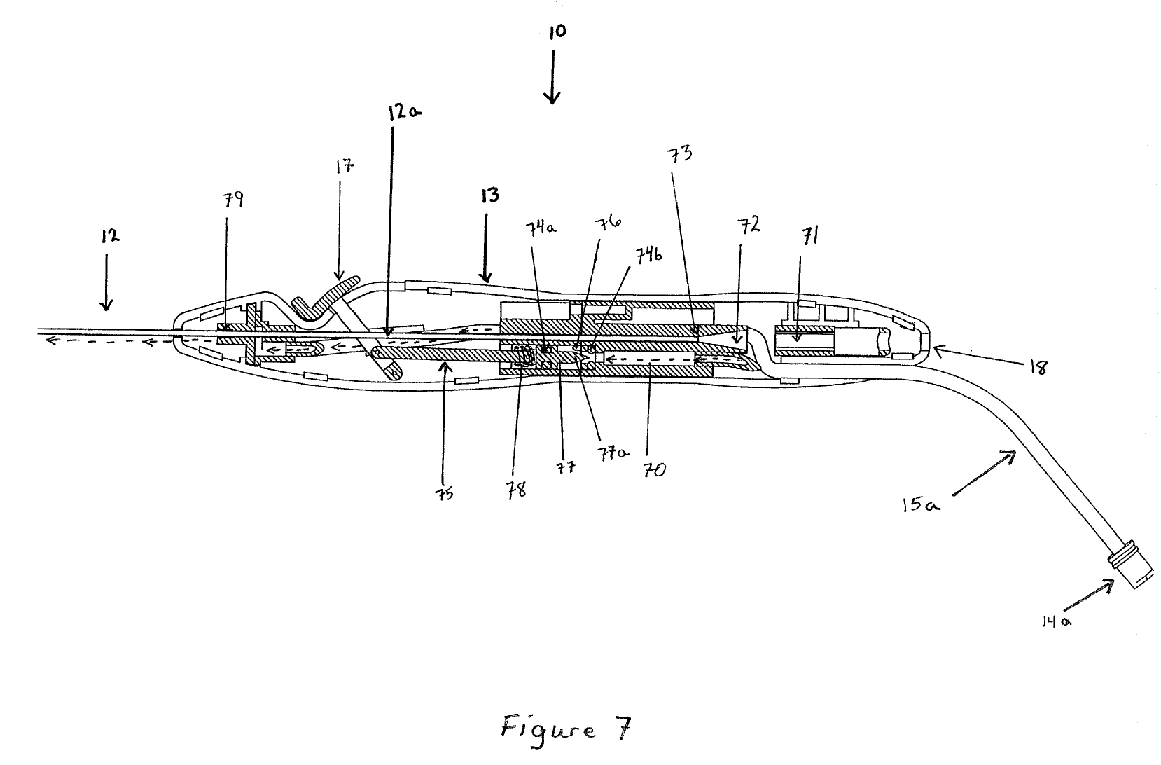

FIG. 7 shows a longitudinal cross-sectional view of an embodiment of the invention. Tissue separator 10 can be seen with shaft 12 connected to handle 13, together with a first fluid supply line 15a and female Leur connector 14a provided to handle 13. The CO₂ flow route and its directionality 70 are shown by the dotted line and arrows, wherein CO₂ or other gas flows through handle 13, continues via the CO₂ cross-over 76, and out side ports 79, into shaft 12, on continuing to head 11, not shown. The CO₂ flow is controlled by linear flow valve 17. Flow valve 17 is mechanically connected to CO₂ valve link 75, which is in turn mechanically connected to piston 77 having a cone portion 77a and a mechanical return mechanism, such as compression spring 78. When flow valve 18 is engaged in a full on position, as shown, spring 78 is compressed and the CO₂ flow proceeds along flow rout 70 at maximum capacity. Depending on the degree of engagement of flow valve 18, CO₂ flow can be varied.

A plurality of O-rings 74a and 74b help maintain a tight seal around piston 77 and cone portion 77a, to prevent CO₂ leaks. An endoscope, not shown, can be inserted into endoscope port 18, through funnel guide 72, and then into a stainless steel tube 12a within. If present in shaft 12, the stainless steel tube 12a runs through handle 13 as well, and terminates within handle 13 at position 73. The endoscope is secured within handle 13 by endoscope lock 71.

Surgical instrument 10 may use a custom designed endoscope or a disposable or reusable endoscope from a variety of manufacturers.

In another embodiment of the invention, surgical instrument 10 may be configured without an endoscope, allowing the physician to decide if an endoscope is necessary for the particular procedure. A plug may be used instead of an endoscope to reduce the cost of the procedure. The plug is configured to form a seal with the endoscope port 18. In another embodiment, the plug may also comprise a length of plastic or metallic material having substantially the same diameter and length of an endoscope in order to provide additional stiffness to shaft 12.

It will be apparent from the above illustrative descriptions of various embodiments of the present invention that such embodiments are presented by way of example only and are not by any interpretation intended by way of limitation. Those skilled in the art could readily devise alternative embodiments and improvements on these embodiments, as well as additional embodiments, without departing from the spirit and scope of the present invention. For example, although a control wire has been described for deploying or retracting the grasping device, a collar may be attached to the endoscope end that engages the grasping device. The grasping device may then be deployed or retracted by unlocking the endoscope lock on the handle and moving the endoscope forward or backward in the shaft. Alternatively, head 11 may be employed in the manner of a spatula. All such modifications are within the scope of the invention as claimed.

What is claimed is:

1. An endoscopic tissue separator surgical instrument comprising:
(a) a shaft having proximal and distal ends;
(b) a head coupled to the distal end of the shaft, the head having an endoscope port and at least two fluid ports, each of the ports characterized by a center; and
(c) a handle coupled to the proximal end of the shaft, the handle including a gas supply port and a fluid supply port in fluid communication with the at least two fluid ports on the head
wherein the centers of the endoscope port and the fluid ports are disposed along an arcuate line of curvature.

2. The endoscopic tissue separator of claim 1, further comprising an endoscope for providing coupling between the distal and proximal ends of the shaft.

3. The endoscopic tissue separator of claim 1, wherein the handle further comprises a flow valve for metering flow of gas between the gas supply port and the at least two fluid ports on the head.

4. The endoscopic tissue separator of claim 1, further comprising a grasping device, the grasping device having a retracted configuration and a deployed configuration wherein the grasping device extends away from the head in the deployed configuration.

5. The endoscopic tissue separator of claim 4, further comprising a deployment control disposed on the handle of the instrument and in mechanical communication with the grasping device.

6. The endoscopic tissue separator of claim 5, wherein the deployment control operates by motion in a direction substantially collinear with the shaft.

7. The endoscopic tissue separator of claim 6, wherein the deployment control is a slide.

8. The endoscopic tissue separator of claim 5, wherein the deployment control can be actuated from both a left and a right side of the handle.

9. The endoscopic tissue separator of claim 5, wherein the mechanical communication between the deployment control and the grasping device includes a control wire having a first wire end and a second wire end, the first wire end connected to the grasping device and the second wire end connected to the deployment control.

10. The endoscopic tissue separator of claim 9, wherein the control wire is straight between the deployment control and the shaft.

11. The endoscopic tissue separator of claim 9, wherein the second wire end is connected to the deployment control by means of a mechanical capture.

12. The endoscopic tissue separator of claim 1, wherein the shaft is a multi-lumen shaft.

13. The endoscopic tissue separator of claim 12, wherein at least one lumen of the shaft is in fluid communication with a fluid supply line for coupling to a saline source.

14. The endoscopic tissue separator of claim 13, wherein at least one lumen of the shaft is in fluid communication with a gas supply line for coupling to a CO₂ source.

15. The endoscopic tissue separator of claim 13, wherein at least one lumen of the shaft houses a tube with an inside diameter of sufficient size to accept an endoscope.

16. The endoscopic tissue separator of claim 5, wherein the grasping device is an extractor for removing tissue during surgical procedures.

17. The endoscopic tissue separator of claim 16, wherein the extractor comprises at least one barb.

18. The endoscopic tissue separator of claim 16, wherein the extractor comprises at least one hook.
